(19) United States
(12) Patent Application Publication
(10) Pub. No.: US 2003/0161853 A1
(43) Pub. Date: Aug. 28, 2003

(54) Methylglycinediacetic acid and deribatives thereof useful as exfoliating agents

(75) Inventors: Elisabeth Picard-Lesboueyries, Velizy (FR); Dominique Bernard, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/345,142

(22) Filed: Jan. 16, 2003

Related U.S. Application Data
(60) Provisional application No. 60/350,375, filed on Jan. 24, 2002.

(30) Foreign Application Priority Data
Jan. 17, 2002 (FR) 0200549

(51) Int. Cl. A61K 7/00

(52) U.S. Cl. 424/401

(57) ABSTRACT

The invention relates to compounds corresponding to formula (I) below:

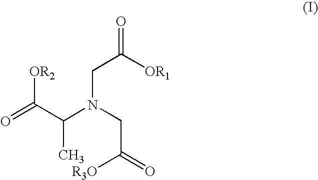

which are useful for example in cosmetic compositions for promoting exfoliation of the skin and/or for stimulating renewal of the epidermis.

### REFERENCE TO PRIOR APPLICATIONS

This application claims priority to provisional U.S. application No. 60/350,375, filed Jan. 24, 2002, and to French patent application 0200549, filed Jan. 17, 2002, both incorporated herein by reference.

### SUMMARY OF THE INVENTION

The invention relates to methylglycinediacetic acid and derivatives thereof, preferably compounds corresponding to formula (I):

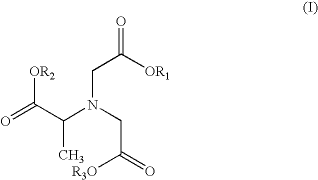

in which:

R₁, R₂ and R₃ independently of one another represent a hydrogen atom; an alkyl group comprising from 1 to 6 carbon atoms and optionally substituted by a radical —COOR₄, where R₄ is an alkyl group comprising from 1 to 4 carbon atoms, or by an amino group, a hydroxyl group, an alkoxy group and/or an alkylamino group; or a sugar residue, and salts thereof, preferably salts of such compounds if R₁ and/or R₂ and/or R₃ represent a hydrogen atom. These compounds are useful for promoting exfoliation of the skin and/or for stimulating renewal of the epidermis. The compounds of the invention can thus be used to, e.g., prevent or treat dry skin and/or signs of skin ageing and/or skin pigmentation.

### BACKGROUND OF THE INVENTION

Exfoliation is a natural phenomenon associated with the fact that the epidermis, which constitutes the top layer of the skin, is constantly regenerating.

The human epidermis consists of several strata of cells in which there are four main types of cells: keratinocytes, which are very much in the majority, melanocytes, Langerhans' cells and Merkel's cells. The stratified character of the epidermis is explained by the distribution of these cells over several superimposed layers.

The epidermis is conventionally divided into a basal layer of keratinocytes constituting the germinal layer of the epidermis, a prickle cell layer consisting of several layers of polyhedral cells arranged on the germinal cells, a granular layer consisting of flattened cells containing distinct cytoplasmic inclusions, namely grains of keratohyalin, and finally a top layer, called the horny layer (or stratum corneum), consisting of keratinocytes at the terminal stage of their differentiation, which are called corneocytes. Corneocytes are atrophied anuclear cells which are derived from the keratinocytes and are eliminated by exfoliation. This surface loss is compensated by the migration of cells from the basal stratum towards the surface of the epidermis, affording perpetual renewal of the epidermis. Forced elimination of the horny layer accelerates renewal and makes it possible to combat skin ageing.

Corneocytes are mainly composed of a fibrous matrix containing cytokeratins, surrounded by a 15 nm thick, very strong structure called the corneocyte envelope. Stacking of these corneocytes constitutes the horny layer responsible for the barrier function of the epidermis. In the course of the normal exfoliation process, the corneocytes nearest the surface become detached from the surface of the epidermis.

Intercellular structures derived from desmosomes, called corneosomes or corneodesmosomes, have been described in the horny layer. Recent studies have shown their major importance in intercorneocyte cohesion and in the exfoliation process.

Corneodesmosine, characterized elsewhere in patent application EP-A-0 972 042, incorporated herein by reference, is a protein of the horny layer of the epidermis which is involved in intercorneocyte cohesion and is a constituent of corneodesmosomes.

In the horny layer a close correlation exists between cell dissociation and the proteolysis of certain corneodesmosomal components such as desmoglein I and corneodesmosine. Several serine proteases of the trypsin or chymotrypsin type seem to be involved in the proteolysis of corneodesmosomes, particularly so-called chymotrypsin-like or trypsin-like proteases (Lundström A., Egelrud T., The Journal of Investigative Dermatology; 1988, 91:340-343 and 1990, 84:216-220).

A variety of agents for combating skin ageing, particularly by promoting exfoliation, i.e. elimination of the “dead” cells situated on the surface of the horny layer of the epidermis, are known in the prior art. Often incorrectly, this “exfoliating” property is also called a keratolytic property.

Thus patent U.S. Pat. No. 4,603,146 describes the use of retinoic acid and its derivatives in cosmetic compositions for combating skin ageing.

Furthermore, numerous patents and publications (cf., for example, patent application EP-A-413 528) and numerous commercial cosmetic compositions teach the use of α-hydroxy acids, such as lactic acid, glycolic acid or citric acid, for the treatment of skin ageing.

Finally, β-hydroxy acids, and more especially salicylic acid and its derivatives, are known for their exfoliating properties (cf. documents WO-A-93/10756 and U.S. Pat. No. 4,767,750).

Nevertheless, the fact remains that the desire to preserve a youthful appearance still leads to the incessant search for novel compounds and/or novel compositions for maintaining or improving the appearance of the skin.

Certain cosmetic active ingredients are capable of stimulating degradation of the corneodesmosomal proteins and hence exfoliation, doubtless by promoting the activity of proteases involved in this process, as seen above.

In this connection, it has been described in patent application EP-A2-0 852 949 (Shiseido) that derivatives of alpha-amino acids of the glycine type promote the degradation of desmoglein (corneodesmosomal protein).

Now, the inventors have discovered, among other things, that sodium methylglycine diacetate is capable of degrading corneodesmosines and thus has a variety of applications in cosmetics and dermatology.

Hitherto, to the inventor's knowledge, this compound has been marketed especially as a metal-chelating agent for incorporation into detergent compositions (detergents) and cosmetic products for the purpose of avoiding the phenomena of rancidity, colour change and precipitation due to heavy metals. JP-10 279 987 thus discloses an antibacterial cleansing composition comprising derivatives of methylglycine diacetic acid.

However, it has never been described in the prior art that sodium methylglycine diacetate is capable of promoting exfoliation and/or stimulating renewal of the epidermis, in particular by degrading the corneodesmosines, and consequently of having applications in the treatment of signs of skin ageing, dry skin and hyperpigmentation.

It is in fact known from WANG et al., Medical Hypotheses, Vol. 53, No. 5, pages 380-382 (1999) that calcium chelating agents are useful as desquamating agents. However, it has been demonstrated that this property is not shared by all chelating agents, so it was not obvious that derivatives of methylglycine diacetic acid, which are known as calcium chelating agents (U.S. Pat. No. 5,481,018) would be effective for desquamating skin.

### DETAILED DESCRIPTION OF THE INVENTION

The present invention relates in a preferred embodiment to compounds corresponding to formula (I) below:

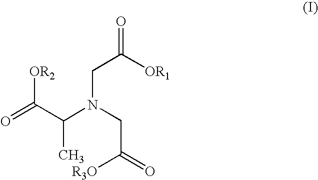

in which:

R₁, R₂ and R₃ independently of one another represent a hydrogen atom; an alkyl group comprising from 1 to 6 carbon atoms optionally substituted by a radical —COOR₄, where R₄ is an alkyl group comprising from 1 to 4 carbon atoms, or by an amino group, a hydroxyl group, an alkoxy group and/or an alkylamino group; or a sugar residue, and salts thereof, preferably salts of such compounds if R₁ and/or R₂ and/or R₃ represent a hydrogen atom. Salts may be inorganic or organic, and include for example: nitrate, sulphate, halogen, (chloride, fluoride, etc.) carbonate, bicarbonate, hydroxide, peroxide, nitride, sulphide, bisulfide, persulphate, glycerophosphate, hypophosphate, borate, citrate, oxalate, acetate, formate, succinate, folinate, aspartate, phthalate, oleate, palmitate, stearate, lauryl sulphate, lanolate, myristate, behenate, caseinate, cyclamate, pantothenate, polyaminopolycarboxylate, thioglycolate, laurate, ricinoleate, pidolate, sorbate and gltcyrrhizinate salts.

These compounds are useful in, for example, a cosmetic composition preferably comprising a physiologically acceptable medium, which may be used for promoting exfoliation of the skin and/or for stimulating renewal of the epidermis.

The compounds of formula (I) above, including its salts, can thus be used in a composition as an agent for preventing or treating dry skin and/or signs of skin ageing and/or skin pigmentation. The invention therefore also relates in a preferred embodiment to these cosmetic uses of the compounds of formula (I).

The invention further relates to a method of treatment for dry skin and/or signs of skin ageing and/or skin pigmentation, which comprises the topical application to the skin of a composition comprising, preferably in a physiologically acceptable medium, at least one compound of formula (I) described above (including salts).

The compounds according to this invention comprise both the individual enantiomers of the compound of formula (I) and all mixtures of these enantiomers.

Examples of alkyl groups useful in formula (I) above include methyl, ethyl, propyl, isopropyl, butyl, isobutyl, sec-butyl, tert-butyl, pentyl and hexyl groups.

As used herein, the term “sugar residue” is understood as meaning the fraction of the sugar molecule to which a hydroxyl group is covalently bonded when the sugar is in the free form. In formula (I) above, this gives a bond of the ester type with the sugar. The latter can be preferably selected from glucose, galactose, xylose, fucose, mannose and polysaccharides. In the case of glucose, for example, the bond of the type —CO—O-[sugar residue] will be:

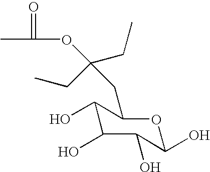

The preferred compounds for use in the present invention are those in which R₁═R₂═R₃═H and their physiologically acceptable salts, in particular their inorganic salts. The particularly preferred compound is sodium methylglycine diacetate, which is the trisodium salt of the compound of formula (I) as defined above (hereinafter trisodium methylglycine diacetate). Such a compound is available especially from BASF under the trade name TRILON M®.

Compositions containing at least one compound of formula (I) preferably contain an effective amount thereof for achieving the desired effect (e.g., treatment of dry skin and/or signs of skin ageing and/or skin pigmentation). By way of example, the composition will preferably contain from 0.01 to 40% by weight and more preferably from 0.1 to 10% by weight of compound(s) of formula (I), based on the total weight of the composition.

The compositions according to the invention are preferably well suited for topical application onto skin and thus preferably comprises a physiologically acceptable medium, i.e. a medium which is compatible with skin and optionally with the mucous membranes and/or hair.

Invention compositions can be provided in any form, and can have for example a greater or lesser fluidity and the appearance of a white or coloured cream, an ointment, a milk, a lotion, a serum, a paste or a foam. It can optionally be applied to the skin in the form of an aerosol or a patch. It can also take the form of a solid, particularly a lipstick. It can be used as a care product and/or as a make-up product for the skin or lips.

Compositions according to the invention can also contain customary adjuvants used in the field of cosmetics, such as hydrophilic or lipophilic gelling agents, hydrophilic or lipophilic active ingredients, preservatives, antioxidants, solvents, perfumes, fillers, filters, pigments, odour absorbers and colorants. The amounts of these various adjuvants are those used in the field in question, for example from 0.01 to 20% of the total weight of the composition. Depending on their nature, these adjuvants can be introduced into the fatty phase or into the aqueous phase. Whatever the case may be, these adjuvants, and their proportions, can be chosen by one of ordinary skill in the art so as not to detract from the desired properties of the compound according to the invention.

If the composition of the invention is an emulsion, the proportion of fatty phase can range from 5 to 80% by weight and preferably from 5 to 50% by weight, based on the total weight of the composition. The oils, emulsifiers and coemulsifiers used in the composition in the form of an emulsion are selected from those conventionally used in the field in question. The emulsifier and coemulsifier are generally present in the composition in a proportion ranging from 0.3 to 30% by weight and preferably from 0.5 to 20% by weight, based on the total weight of the composition.

The following is a non-limiting list of examples of oils that can be used in the composition of the invention:

hydrocarbon oils of animal origin, such as perhydrosqualene;

hydrocarbon oils of vegetable origin, such as liquid triglycerides of fatty acids containing from 4 to 10 carbon atoms, and the liquid fraction of shea butter;

synthetic esters and ethers, especially those of fatty acids, such as oils of the formulae R¹COOR² and R¹OR², in which R¹ represents the radical of a fatty acid containing from 8 to 29 carbon atoms and R² is a branched or unbranched hydrocarbon chain containing from 3 to 30 carbon atoms, for example Purcellin oil, isononyl isononanoate, isopropyl myristate, 2-ethylhexyl palmitate, 2-octyldodecyl stearate, 2-octyldodecyl erucate and isostearyl isostearate; hydroxylated esters such as isostearyl lactate, octyl hydroxystearate, octyldodecyl hydroxystearate, diisostearyl malate, triisocetyl citrate and fatty alcohol heptanoates, octanoates and decanoates; polyol esters such as propylene glycol dioctanoate, neopentyl glycol diheptanoate and diethylene glycol diisononanoate; and pentaerythritol esters such as pentaerythrityl tetraisostearate;

linear or branched hydrocarbons of mineral or synthetic origin, such as volatile or non-volatile paraffin oils and derivatives thereof, petrolatum, polydecenes, and hydrogenated polyisobutene such as parleam;

fatty alcohols having from 8 to 26 carbon atoms, such as cetyl alcohol, stearyl alcohol and their mixture (cetylstearyl alcohol), octyldodecanol, 2-butyloctanol, 2-hexyldecanol, 2-undecylpentadecanol, oleyl alcohol and linoleyl alcohol;

fluorinated oils with a partially hydrocarbon and/or silicone structure, such as those described in document JP-A-2-295912;

silicone oils such as volatile or non-volatile polymethylsiloxanes (PDMS) with a linear or cyclic silicone chain, which are liquid or pasty at room temperature, especially cyclopolydimethylsiloxanes (cyclomethicones) such as cyclohexasiloxane; polydimethylsiloxanes containing alkyl, alkoxy or phenyl groups that are pendant or at the end of the silicone chain, said groups having from 2 to 24 carbon atoms; and phenylsilicones such as phenyltrimethicones, phenyldimethicones, phenyltrimethylsiloxydiphenylsiloxanes, diphenyldimethicones, diphenylmethyldiphenyltrisiloxanes, 2-phenylethyltrimethylsiloxy silicates and polymethylphenylsiloxanes; and

mixtures thereof.

The following is a non-limiting list of examples of emulsifiers and coemulsifiers which can be used in the invention: O/W emulsifiers such as fatty acid esters of polyethylene glycol, especially PEG- 100 stearate, and esters of fatty acids and of glycerol, such as glyceryl stearate, and also W/O emulsifiers such as the ethoxylated poly(methylcetyl)(dimethyl)methylsiloxane available under the trade name ABIL WE09 from Degussa Goldschmidt.

Useful hydrophilic gelling agents include carboxyvinylic polymers (carbomers), acrylic copolymers such as acrylate/alkylacrylate copolymers, polyacrylamides, polysaccharides, natural gums and clays, and lipophilic gelling agents which may be mentioned are modified clays such as bentones, fatty acid metal salts, hydrophobic silica and polyethylenes.

The following non-limiting list may be mentioned, in addition to pigments, as examples of fillers which can be used in the composition of the invention: silica powder; talcum, starch crosslinked with octenylsuccinic anhydride, marketed by National Starch under the name DRY FLO PLUS (28-1160); polyamide particles, especially those sold under the name ORGASOL by Atochem; polyethylene powders; microspheres based on acrylic copolymers, such as those of ethylene glycol dimethacrylate/lauryl methacrylate copolymer sold by Dow Corning under the name POLYTRAP; expanded powders such as hollow microspheres, especially the microspheres marketed under the name EXPANCEL by Kemanord Plast or under the name MICROPEARL F 80 ED by Matsumoto; silicone resin microbeads such as those marketed under the name TOSPEARL by Toshiba Silicone; and mixtures thereof. These fillers can be present in any amount, for example amounts ranging from 0 to 20% by weight and preferably from 1 to 10% by weight, based on the total weight of the composition or the preparation according to the invention.

UVA and/or UVB filters, selected from organic filters and inorganic filters optionally coated for hydrophobicity, can also be introduced into the composition according to the invention.

Useful active ingredients which can be used in the composition according to the invention include: depigmenting or propigmenting agents; antiglycating agents; NO synthase inhibitors; 5α-reductase inhibitors; lysyl and/or prolyl hydroxylase inhibitors; agents stimulating the synthesis of dermal or epidermal macromolecules and/or preventing their degradation; agents stimulating fibroblast and/or keratinocyte proliferation or stimulating keratinocyte differentiation; muscle relaxants; antimicrobials; tensors; pollution inhibitors and/or free radical inhibitors; soothing agents; agents with a lipolytic activity or a direct or indirect favourable activity on adipose tissue reduction; agents acting on the microcirculation; agents acting on the cells' energy metabolism; and mixtures thereof.

The invention will be better understood and its advantages will become more clearly apparent from the following Examples, which are given by way of illustration and without implying a limitation.

### EXAMPLES

### Example 1

Exfoliation Test by Measurement of the Degradation of Corneodesmosines

This Example studies the capacity of trisodium methylglycine diacetate to promote exfoliation by the degradation of corneodesmosines.

Corneodesmosine is one of the majority markers of exfoliation in the corneodesmosome. It is studied by immunoblotting after separation by electrophoresis and transfer to a membrane. After specific labelling with the monoclonal antibody G36-19, it is revealed by chemiluminescence. The murine monoclonal antibody G36-19 is specific for corneodesmosine; it belongs to a series of antibodies directed against epidermal differentiation antigens, produced after the immunization of a mouse with a human plantar horny layer homogenate, and then characterized (Serre G. et al, J. Invest. Dermatol. 1991, 97(6), 1061-72).

Varnish strippings are performed on the lower legs of volunteers (modification of the procedure of Lundström A. and Egelrud T., Acta Derm. Venereol. (stockh) 71, 471-474, 1991). The nylon-varnish leaves associated with the corneocytes are immersed in acetone (1 ml/cm²) to detach the corneocytes. The mixture is filtered then rinsed three times with the same volume of acetone in order to remove all traces of varnish. Finally, the mixture is dried under vacuum:

acetone-containing powders of stratum corneum are thus obtained.

The acetone-containing powders are divided up into 1 mg aliquots. 100 μl of aqueous solutions containing 2% of active ingredient, adjusted to pH 8.0, are added. Controls without active ingredient are prepared under the same conditions. Two incubation times are studied: t=0 and t=8 h. In the latter case, incubation takes place at 34° C. with agitation.

After incubation, the mixtures are centrifuged for 10 min at 10,000 g. The supernatant is removed and replaced with 100 μl of Laemmli buffer (0.0625 M Tris/HCl pH 6.8, 2% SDS, 200 mM DTT, 10% glycerol) for extraction of the proteins. The mixture is boiled for 10 min at 100° C. and then ground in a Potter mill. The mixture is centrifuged for 10 min at 10,000 g and the supernatant is then collected. It contains the corneodesmosomal proteins.

The total proteins are assayed by Bradford's method (Biorad kit). This allows an adjustment of the samples to 0.6 mg/ml and a true comparison of the treatments. The samples together with a Rainbow low-molecular standard (Amersham Pharmacia Biotech), in a ratio of ⅓, are separated by electrophoresis on 12% acrylamide gel for ½ h at 100 V and then for 1 h at 200 V. After electrophoresis, the proteins are transferred to an Immobilon-P membrane (Millipore) for 3 h at 60 V. The membrane is then incubated for 2 times 15 min in TBS-TL buffer (Tris 25 mM, 0.15 M NaCl pH 7.2, 0.05% Tween 20, 0.5% skimmed milk powder) to block the non-specific sites. Incubation with the antibody G36-19, in a ratio of 1/12,500, is performed overnight at 4° C. After two rinses of 5 min in TBS-TL, the membrane is incubated with an IG(H+L) anti-mouse goat antibody/peroxidase conjugate (Biorad), in a ratio of 1/4000, for 1 h 30 min at room temperature. After several rinses of 5 min in TBS-TL and then TBS (without milk or Tween), the membrane is incubated for 1 min in 10 ml of ECL reagent (Amersham Pharmacia Biotech). The chemiluminescence of the corneodesmosine bands is measured with FluorS Multimager (Biorad). The 46 kDa band is quantified using the Quantity-one software (Biorad).

The results of this study are summarized in Table 1 below.

Glycine is used as a reference compound in this study, it having been shown in patent application EP-A2-0 852 949 (Shiseido) that glycine promotes the degradation of desmoglein (corneodesmosomal protein). It was tested according to a protocol similar to that described above.

TABLE 1
Effect of trisodium methylglycine diacetate on the degradation of
corneodesmosines

| | Percentage increase in the |
|---|---|
| Test molecules | degradation of corneodesmosine |
| Control | 0% |
| Trisodium methylglycine diacetate | 59% |
| TRILON M ® from BASF) | |
| Glycine | 30% |

The control is prepared under the test conditions with the solubilizing buffer and without active ingredient. This control takes account of the natural degradation of corneodesmosines which takes place during incubation.

It is clearly apparent that trisodium methylglycine diacetate promotes the degradation of corneodesmosines and that it is better than the glycine used as positive control in this test.

### Example 2

Comparative Test

Two other chelating agents were tested in the same manner as that described in Example 1: sodium iminosuccinate and lauroylethylenediaminetriacetic acid.

These compounds did not have a favourable activity on the degradation of corneodesmosine, as shown in Table 2 below.

TABLE 2
Effect of two chelating agents on the degradation of corneodesmosines

| | % increase in the degradation |
|---|---|
| Test molecules | of corneodesmosine |
| Control | 0% |
| Lauroylethylenediaminetriacetic acid | −80% |
| Na iminosuccinate | −79% |

### Example 3

Proexfoliating Moisturizing Cream

The following composition is prepared in a manner conventional to those skilled in the art:

| Phase A | | |
|---|---|---|
| Demineralized water | qsp | 100% |
| Preservatives | | 0.5% |
| Carbomer | | 0.4% |
| Glycerol | | 7% |
| Phase B1 | | |
| Ethoxylated sorbitan stearate (200 EO) | | 0.9% |
| Phase B2 | | |
| PEG-100 stearate and glyceryl stearate | | 2.1% |
| Apricot oil | | 10% |
| Petrolatum | | 2% |
| Parleam | | 10% |
| IN filter | | 1% |
| Ceramides | | 0.5% |
| Phase C | | |
| Water | | 2% |
| Hydrochloric acid | qs ppH | 7 |
| Trisodium methylglycine diacetate | | 5% |

This cream can be used to care for dry skin.

### Example 4

Proexfoliating Anti-Ageing Cream

| Phase A | | |
|---|---|---|
| Acrylate/C₁₀₋₃₀-acrylate copolymer | | 0.5% |
| Water | | 12% |
| Phase B | | |
| Hydrogenated polyisobutene | | 5% |
| Cyclohexasiloxane | | 5% |
| Octyl methoxycinnamate | | 1% |
| Phase C | | |
| Trisodium methylglycine diacetate | | 1% |
| Hydrochloric acid | qsp pH | 7 |
| Glycerol | | 6% |
| Preservatives | | 0.5% |
| Centella asiatica extract | | 1% |
| Water | qsp | 100% |
| Phase D | | |
| Polyacrylamide and C₁₃₋₁₄-isoparaffin and laureth-7 | 1% | |

This composition can be prepared in the following manner. The polymer of phase A is dispersed in water at 40° C. The constituents of phase B are heated at 70° C. until they have completely dissolved, after which the temperature is brought down to 40° C. The constituents of phase C are mixed at 50° C. Phase B is then introduced into phase A at 40° C., with agitation, followed by phase C and gelling phase D.

### Example 5

Depigmenting Cream

The following composition is prepared in a manner conventional to those skilled in the art.

| Phase A | | |
|---|---|---|
| Demineralized water | qsp | 100% |
| Preservatives | | 0.5% |
| Carbomer | | 0.4% |
| Glycerol | | 7% |
| Phase B1 | | |
| Ethoxylated sorbitan stearate (200 EO) | | 0.9% |
| Phase B2 | | |
| PEG-100 stearate and glyceryl stearate | | 2.1% |
| Cyclohexasiloxane | | 10% |
| Parleam | | 5% |
| UV filter | | 2% |
| Phase C | | |
| Water | | 2% |
| Hydrochloric acid | qsp pH | 7 |
| Trisodium methylglycine diacetate | | 5% |
| Kojic acid | | 0.1% |

This cream can be used to reduce liver spots on the hands and on the neck and shoulders.

All documents, tests, patents, applications, references, articles, publications, etc. mentioned above are incorporated herein by reference.

The above description sets forth the manner and process of making and using the present invention and enables any person skilled in the art to which it pertains to make and use the same. Preferred embodiments of the invention so enabled include the use of at least one compound corresponding to formula (I) above (including salts), for example in a composition comprising a physiologically acceptable medium, as an agent for promoting exfoliation of the skin and/or for stimulating renewal of the epidermis, and for treatment for dry skin and/or signs of skin ageing and/or skin pigmentation. Similarly described and enabled are the embodiments of the invention set forth in the following claims, which make up a part of this disclosure.

1. A method of promoting exfoliation of the skin and/or for stimulating renewal of the epidermis comprising applying to skin in need thereof an amount of at least one compound of formula (I): 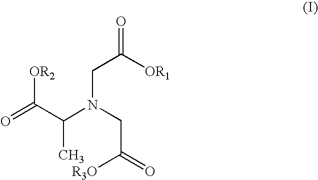
in which:
R₁, R₂ and R₃ independently of one another represent a hydrogen atom; an alkyl group comprising from 1 to 6 carbon atoms and optionally substituted by a radical —COOR₄, where R₄ is an alkyl group comprising from 1 to 4 carbon atoms, or by an amino group, a hydroxyl group, an alkoxy group and/or an alkylamino group; or a sugar residue, and salts thereof, sufficient to exfoliate the skin and/or stimulate renewal of the epidermis.

2. The method according to claim 1, wherein the compound of formula (I) is selected from those compounds where R₁═R₂═R₃═H and their salts.

3. The method of claim 1, wherein said salts are inorganic salts.

4. The method of claim 2, wherein said salts are inorganic salts.

5. The method of claim 1, comprising applying trisodium methylglycinediacetate to skin in need thereof in an amount sufficient to exfoliate the skin and/or stimulate renewal of the epidermis.

6. The method of claim 1, wherein at least one of R₁, R₂ and R₃ is a sugar residue selected from the group consisting of glucose, galactose, xylose, fucose, mannose and polysaccharides.

7. The method of claim 1, wherein said at least one compound of formula (I) is present in a composition comprising said at least one compound and a physiologically acceptable medium.

8. The method according to claim 7, wherein said composition contains from 0.1 to 10% by weight of compound of formula (I), based on the total weight of the composition.

9. The method of claim 5, wherein said trisodium methylglycine diacetate is present in a composition comprising said trisodium methylglycine diacetate and a physiologically acceptable medium.

10. The method according to claim 9, wherein said composition contains from 0.1 to 10% by weight of trisodium methylglycine diacetate, based on the total weight of the composition.

11. A method of preventing or treating dry skin and/or signs of skin ageing and/or skin pigmentation comprising applying to skin in need thereof an amount of at least one compound of formula (I): 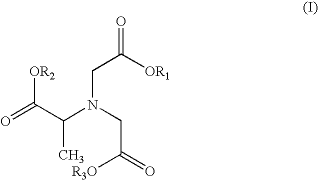
in which:
R₁, R₂ and R₃ independently of one another represent a hydrogen atom; an alkyl group comprising from 1 to 6 carbon atoms and optionally substituted by a radical —COOR₄, where R₄ is an alkyl group comprising from 1 to 4 carbon atoms, or by an amino group, a hydroxyl group, an alkoxy group and/or an alkylamino group; or a sugar residue, and salts thereof, sufficient to prevent or treat dry skin and/or signs of skin ageing and/or skin pigmentation.

12. The method according to claim 11, wherein the compound of formula (I) is selected from those compounds where R₁═R₂═R₃═H and their salts.

13. The method of claim 11, wherein said salts are inorganic salts.

14. The method of claim 12, wherein said salts are inorganic salts.

15. The method of claim 11, comprising applying trisodium methylglycine diacetate to skin in need thereof in an amount sufficient to prevent or treat dry skin and/or signs of skin ageing and/or skin pigmentation.

16. The method of claim 11, wherein at least one of R₁, R₂ and R₃ is a sugar residue selected from the group consisting of glucose, galactose, xylose, fucose, mannose and polysaccharides.

17. The method of claim 11, comprising applying trisodium methylglycine diacetate to skin in need thereof in an amount sufficient to prevent or treat dry skin and/or signs of skin ageing.

18. The method of claim 11, wherein said at least one compound of formula (I) is present in a composition comprising said at least one compound and a physiologically acceptable medium.

19. The method according to claim 18, wherein said composition contains from 0.1 to 10% by weight of compound of formula (I), based on the total weight of the composition.

20. The method of claim 15, wherein said trisodium methylglycine diacetate is present in a composition comprising said trisodium methylglycine diacetate and a physiologically acceptable medium.

21. The method according to claim 20, wherein said composition contains from 0.1 to 10% by weight of trisodium methylglycine diacetate, based on the total weight of the composition.
